(19) United States
(12) Patent Application Publication
(10) Pub. No.: US 2019/0374363 A1
(43) Pub. Date: Dec. 12, 2019

(54) ANKLE BRACE

(71) Applicant: Melanie Deborah Hatto, Cape Coral, FL (US)

(72) Inventor: Melanie Deborah Hatto, Cape Coral, FL (US)

(21) Appl. No.: 16/437,357

(22) Filed: Jun. 11, 2019

Related U.S. Application Data
(60) Provisional application No. 62/683,095, filed on Jun. 11, 2018.

(51) Int. Cl. A61F 5/01

(52) U.S. Cl. CPC A61F 5/0111

(57) ABSTRACT

An ankle brace includes at least three straps including a first strap, a second strap, and at least a third strap that is elongated in length as compared to a length of the first and the second strap. The first and second straps are parallel to one another and secured to one another by at least one inter-strap fastener, and each have inner padding and an outer attachment element on at least one end, wherein the first and the second straps are configured for wrapping around a user's ankle. The third strap is secured on one end to at least one of the first strap and the second strap, and the third strap has an attachment element opposite the secured end for attaching to at least one of the first and the second straps.

### CROSS REFERENCE TO RELATED APPLICATIONS

This Application claims the benefit of provisional application Ser. No. 62/683,095 entitled “ankle brace” filed on Jun. 11, 2018, which is herein incorporated by reference in its entirety.

### FIELD

This Disclosure relates to the field of ankle braces.

### BACKGROUND

Ankle braces (or ankle supports) are commonly worn as a treatment for ankle injuries or to prevent an ankle sprain. A conventional ankle brace protects one's ankle(s) from potentially harmful lateral movement by limiting ankle mobility and helps provide support and protection to weak, unstable or injured ankles, using compression as the main tool of providing support.

### SUMMARY

This Summary is provided to introduce a brief selection of disclosed concepts in a simplified form that are further described below in the Detailed Description including the drawings provided. This Summary is not intended to limit the claimed subject matter's scope.

This Disclosure recognizes conventional ankle braces do not allow the user to adjust the tension to contour the proximal (near side) part of the user's ankle. Moreover, conventional ankle braces do not allow for a relatively narrow width and longer support wrapping under the foot where the tension level can be adjusted by the user for the particular user's ankle condition and for the user's comfort. Disclosed ankle braces have at least three straps comprising a first strap, a second strap, and at least a third strap that is elongated as compared to the first and the second strap for accurately supporting various different conditions where an ankle brace is needed.

The first and second straps are parallel to one another and secured to one another by at least one inter-strap fastener, and each have inner padding and an outer attachment element on at least one end, wherein the first and the second straps are configured for wrapping around a user's ankle. The third strap is secured on one end to at least one of the first and the second strap, and the third strap has an attachment element opposite the secured end for attaching to at least one of the first and the second straps. Disclosed ankle braces are believed to be distinctive and advantageous relative to known ankle braces because the elongated third strap is able to be positioned towards the proximal or the distal end of the user's foot for allowing support including for distinct alignment of the Tarsals by supporting selected different ligaments and tendons related to the ankle.

### BRIEF DESCRIPTION OF THE DRAWINGS

Reference will now be made to the accompanying drawings, which are not necessarily drawn to scale, wherein:

FIG. 1 is a depiction of the inside an example ankle brace, according to an example embodiment.

FIG. 2 is a depiction of the ankle brace in FIG. 1 placed on a user's ankle, before being secured to a user's ankle.

FIG. 3 is a depiction of the ankle brace after the first and second straps are positioned to form a first loop on the user's ankle using the attachment elements.

FIG. 4 is a depiction of the ankle brace after the third strap shown in FIG. 3 is wrapped back across the ankle of the user to form a loop.

FIG. 5 is a depiction of the ankle brace after the third strap shown in FIG. 4 is secured to the first and second straps at an angle of between twenty-five degrees and sixty degrees, so that the ankle brace is ready for use protecting a user's ankle.

### DETAILED DESCRIPTION

Disclosed embodiments are described with reference to the attached figures, wherein like reference numerals, are used throughout the figures to designate similar or equivalent elements. The figures are not drawn to scale and they are provided merely to illustrate aspects disclosed herein. Several disclosed aspects are described below with reference to example applications for illustration. It should be understood that numerous specific details, relationships, and methods are set forth to provide a full understanding of the embodiments disclosed herein.

Notwithstanding that the numerical ranges and parameters setting forth the broad scope of this Disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 5.

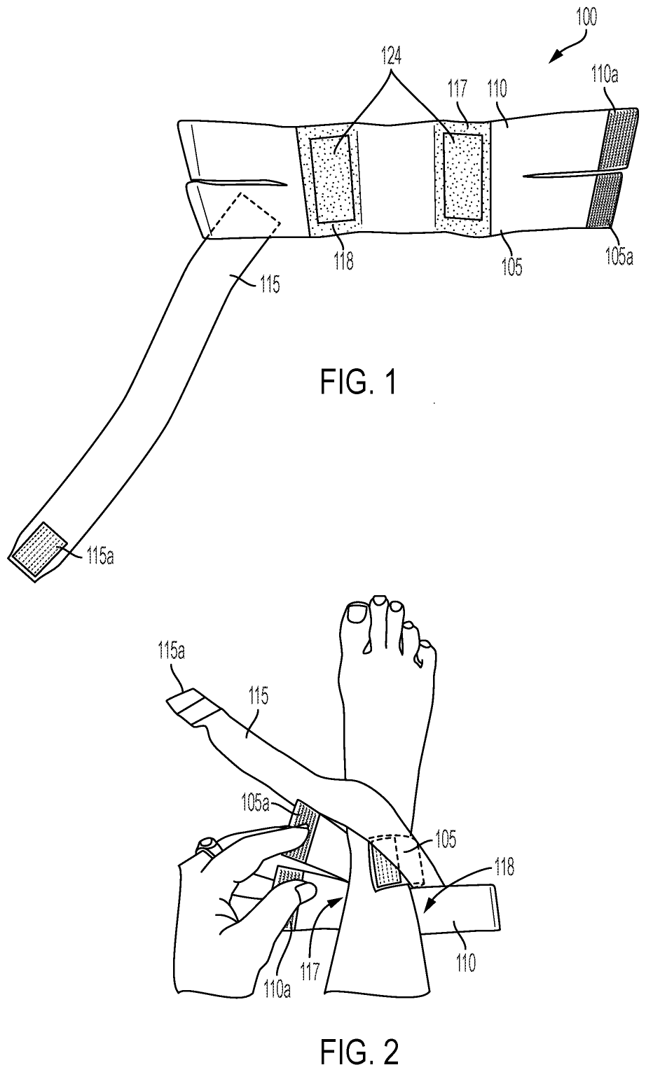

FIG. 1 is a depiction of the inside of an example ankle brace 100, according to an example embodiment. The ankle brace 100 includes at least three straps including a first strap 105, a second strap 110, and at least a third strap 115 that is elongated in length as compared to the first strap 105 and the second strap 110, such as being at least 15% to 45% longer. The respective components of the ankle brace 100 can each comprise a loop fabric material also called a hook compatible material, such as VELCRO® VELTEX® which is a laminated product with a nylon face fabric, polyester foam core, and nylon backing that has loops designed for engagement with a variety of articles that have tiny hooks. Other example loop fabric materials include Engageable Elastic, unbroken loop (UBL) fabric, broken loop fabric, styrenebutadiene rubber (SBR), and polychloroprene (Neoprene).

The first and second straps 105, 110 are parallel to one another and secured to one another by at least one inter-strap fasteners 117, 118 both shown being oriented perpendicular to the length direction of the first and second straps 105, 110. The inter-strap fasteners 117, 118 can comprise elastic strips. There is padding 124 shown over the inter-strap fasteners 117, 118.

The padding 124 can comprise a foam that includes a phase change material. Such materials are known in bedding and are sometimes referred to as a “gel-infused foam” or “cooling foam”. Cooling foam is a type of foam blended with a gel or gel beads made with phase change material(s). The phase change materials can generally be any of a host of different known phase change chemicals. Phase change materials within a foam provide a surface that when in contact with the foam feels cooler because the phase change materials have a property associated with a phase change that transfers heat from the user into cooling foam.

The first and second straps 105, 110 generally include a fastener on at least one end shown as 105a and 110a, respectively. As used herein a ‘strip’ is an article that has a length that is at least six (6) times its width or average width where the width varies along its length. The first and second straps 105, 110 are for wrapping around a user's ankle and for providing an attachment surface (such as being attachable by hook) for the attachment end 115a of the third strap 115

The attachment element 105a, 110a for the first and second straps 105, 110, and 115a for the third strap 115 can comprise hooks in the case of a hook and loop-based attachment. Also, the attachment elements can comprise snaps or buckles. In the case of hook and loop-based attachment elements, hooks will generally be on the attachment ends and loops will be on the other portions of the first and second straps 105 and 110.

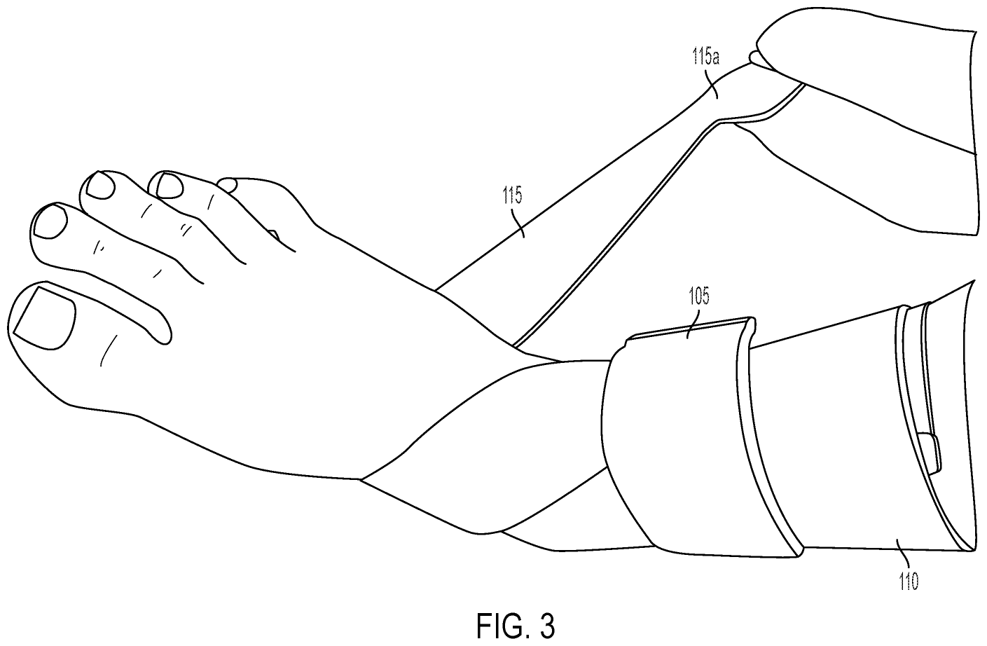

Disclosed attachments can all be between hooks and loops, such as a VELCRO hook to loop, or another hook and loop structure. VELCRO is known to be a commercially available lineal fabric strip with small hooks that can “mate” with another fabric strip with smaller loops, attaching temporarily, until pulled apart by the user. The third strap 115 is generally secured at its attachment end with a stitching or by a hook that and on its attachment and shown herein is 115a (not shown in FIG. 1, but see attachment element 115a shown in FIG. 3 described below) after wrapping disclosed herein generally attaches to a loop structure provided by the first and second straps 105, 110, and at an angle generally between twenty five (25) degrees and sixty (60) degrees relative to the length direction of the first and second straps 105, 110. The attachment element 115a at or near the third strap's 115 unsecured end.

The first and second straps 105, 110 are configured for wrapping around a user's ankle to collectively form a first loop around the user's ankle which is secured in place by their attachment elements 105a, 110a. The third strap 115 is configured for wrapping around a user's foot and ankle to form a second loop that crosses over itself and attaches to the outside of the first strap 105 or the second strap 110 with its attachment element shown below is 115a. The third strap can be placed across a top of a foot of the user and be wrapped underneath and crossing back over a top of the foot to an opposite side of the ankle forming a figure eight (8) pattern, then for attaching to at least one of first and second straps 105, 110. The third strap 115 may have a moveable or slidable pad element, such as if the user's Achilles tendon is bigger or smaller than the pad can be moved slightly to allow it to stay just behind the malleolus's of the ankle.

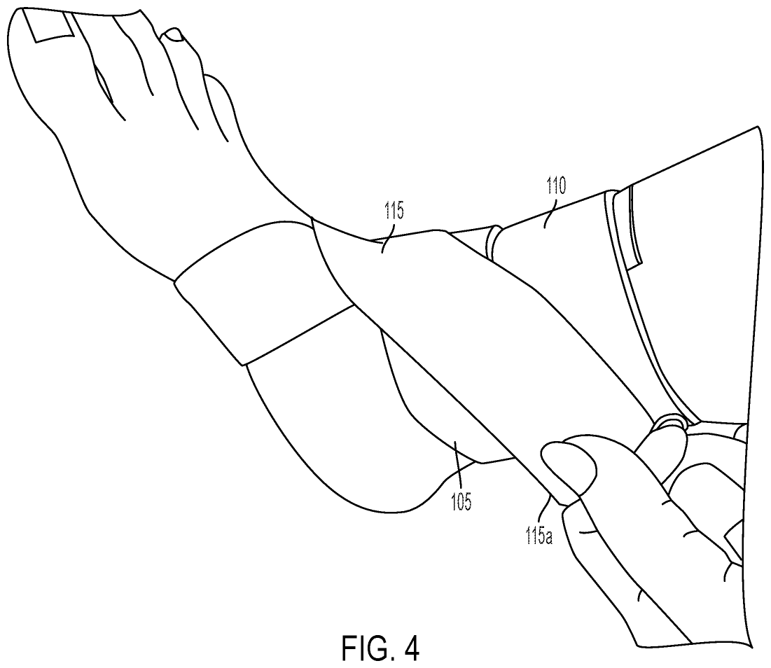

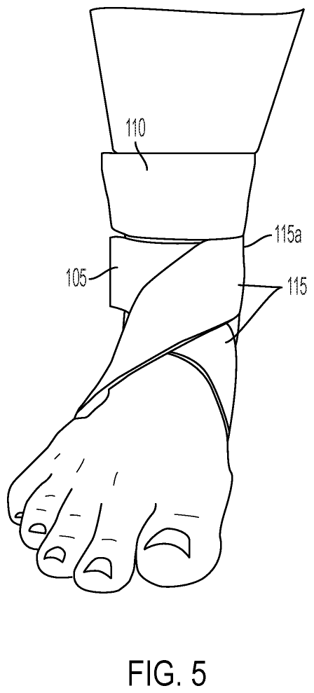

FIG. 2 is a depiction of the ankle brace 100 shown in FIG. 1 after being initially placed on a user's ankle, before being secured to the user's ankle. FIG. 3 is a depiction of the ankle brace 100 after the first and second straps 105, 110 are positioned to collectively form a first loop around a proximal portion of a Fuser's ankle (the portion located closest to the point of attachment of the ankle) using the respective attachment elements 105a, 110a. The third strap 115 is shown having its attachment end 115a and after being wrapped below and partially around the foot, but not yet forming a loop. FIG. 4 is a depiction of the ankle brace 100 after the third strap 115 shown in FIG. 3 is wrapped back across the user's ankle to form a loop. FIG. 5 is a depiction of the ankle brace 100 after the third strap 115 shown in FIG. 4 is shown secured to the first and second straps 105, 110 at an angle of about 45° relative to the length direction of the first and second straps 105, 110, thus being between twenty five degrees and sixty degrees so that the ankle brace 100 is ready for use protecting the user's ankle.

The ankle brace may come with a right (R) or a left (L) label which is for treating common ankle injuries, but the ankle brace regardless of its R or L label can be used on either ankle. For example, if the label is R and the user puts it on their right ankle, then the first strap and second strap will hook towards the outside of the ankle and the third strap 115 will fasten to the inside. If one uses the R labeled ankle brace on their left side, then the first strap 105 and the second strap 110 will attach towards the inside of the user's ankle, and the attachment element 115a of the third strap 115 will attach to the outside surface of first 105 and/or seconds straps 110 around the outside of the user's ankle.

Disclosed aspects also include a method of protecting and ankle using a disclosed ankle brace. The method comprises placing the inner padding 124 of the first strap 105 and the second strap 110 around the user's ankle an ankle and then placing the third strap 115 towards a bottom of a foot of the user. A tension of the first strap 105 and a tension of the second strap 110 are adjusted, then the first strap and the second strap are secured as described above. The third strap 115 is placed across a top of the foot, and is wrapped underneath and crossing back over a top of the foot to an opposite side of the ankle forming a figure eight (8) pattern. A tension of the third strap 115 is adjusted and the third attachment element 115a is secured to at least one of the first strap 105 and the second strap 110.

While various disclosed embodiments have been described above, it should be understood that they have been presented by way of example only, and not as a limitation. Numerous changes to the disclosed embodiments can be made in accordance with the Disclosure herein without departing from the spirit or scope of this Disclosure. Thus, the breadth and scope of this Disclosure should not be limited by any of the above-described embodiments. Rather, the scope of this Disclosure should be defined in accordance with the following claims and their equivalents.

Although disclosed embodiments have been illustrated and described with respect to one or more implementations, equivalent alterations and modifications will occur to others skilled in the art upon the reading and understanding of this specification and the annexed drawings. While a particular feature may have been disclosed with respect to only one of several implementations, such a feature may be combined with one or more other features of the other implementations as may be desired and advantageous for any given or particular application.

1. An ankle brace, comprising:
at least three straps including a first strap, a second strap, and at least a third strap that is elongated in length as compared to a length of the first strap and the second strap;
wherein the first and second straps are parallel to one another and secured to one another by at least one inter-strap fastener, each having inner padding and an outer attachment element on at least one end, wherein the first and the second straps are configured for wrapping around a user's ankle,
wherein the third strap has a third attachment element on at least one end and is configured for being placed across a top of a foot of the user and being wrapped underneath and crossing back over a top of the foot to an opposite side of the ankle forming a figure eight (8) pattern then for attaching to the third attachment element to at least one of the first strap and the second strap.

2. The ankle brace of claim 1, wherein the third strap is configured for being secured opposite the third attachment element to the first strap or the second strap and at an angle between twenty-five degrees and sixty degrees.

3. The ankle brace of claim 1, wherein the inter-strap fastener comprises two spaced apart padded strips that are oriented perpendicular relative to the first strap and to the second strap.

4. The ankle brace of claim 1, wherein all the attachment elements comprise hook structures and wherein the first, the second, and the third straps all comprise a loop fabric material.

5. The ankle brace of claim 1, wherein the length of the third strap is a sufficient length to loop at least once around a foot of the user.

6. The ankle brace of claim 1, wherein the first and second straps are configured to collectively form a loop around a proximal portion of the user's ankle.

7. The ankle brace of claim 1, wherein the inner padding comprises a foam that includes a phase change material.

8. A method, comprising:
placing an ankle brace on an ankle of a user, the ankle brace comprising at least three straps including a first strap, a second strap, and at least a third strap having a third attachment element that is elongated in length as compared to a length of the first strap and the second strap; wherein the first and second straps are secured to one another by at least one inter-strap fastener and each have inner padding and an outer attachment element on at least one end, wherein the first and the second straps are configured for wrapping around a user's ankle,
placing the inner padding of the first strap and the second strap around the user's ankle an ankle and then placing the third strap towards a bottom of a foot of the user;
adjusting a tension of the first strap and the second strap;
securing the first strap and the second strap;
placing the third strap across a top of the foot,
wrapping the third strap underneath and crossing back over a top of the foot to an opposite side of the ankle forming a figure eight (8) pattern,
adjusting a tension of the third strap, and
attaching the third attachment element to at least one of the first strap and the second strap.

9. The method of claim 8, wherein the third strap is secured opposite the third attachment element at an angle to the first strap or the second strap at an angle between twenty-five degrees and sixty degrees.

10. The method of claim 8, wherein the inter-strap fastener comprises two spaced apart padded strips that are oriented perpendicular relative to the first strap and to the second strap.

11. The method of claim 8, wherein all the attachment elements comprise hook structures and wherein the first, the second, and the third straps all comprise a loop fabric material.

12. The method of claim 8, wherein the padding comprises a foam that includes a phase change material.

13. An ankle brace, comprising:
at least three straps including a first strap, a second strap, and at least a third strap that is elongated in length as compared to a length of the first strap and the second strap;
wherein the first and second straps are parallel to one another and secured to one another by at least one inter-strap fastener, each having inner padding and an outer attachment element on at least one end, wherein the first and the second straps are configured for wrapping around a user's ankle,
wherein the third strap has a third attachment element on at least one end and is configured for being placed across a top of a foot of the user and being wrapped underneath and crossing back over a top of the foot to an opposite side of the ankle forming a figure eight (8) pattern then for attaching to the third attachment element to at least one of the first strap and the second strap;
wherein the inter-strap fastener comprises two spaced apart padded strips that are oriented perpendicular relative to the first strap and to the second strap;
wherein all the attachment elements comprise hook structures,
wherein the first, the second, and the third straps all comprise a loop fabric material.
